(19) United States
(12) Patent Application Publication
(10) Pub. No.: US 2020/0118659 A1
(43) Pub. Date: Apr. 16, 2020

(54) METHOD AND APPARATUS FOR DISPLAYING VALUES OF CURRENT AND PREVIOUS STUDIES SIMULTANEOUSLY

(71) Applicant: FUJIFILM MEDICAL SYSTEMS U.S.A., INC., Stamford, CT (US)

(72) Inventors: Adam Hooker, Indianapolis, IN (US); Greg Hoofnagle, Morrisville, NC (US)

(21) Appl. No.: 16/156,852

(22) Filed: Oct. 10, 2018

(51) Int. Cl. G16H 15/00; G06F 3/14; G16H 50/20

(52) U.S. Cl. CPC G16H 15/00; G06T 11/206; G16H 50/20; G06F 3/14

(57) ABSTRACT

A method and a system for displaying values of current and previous studies simultaneously are disclosed. In one embodiment, the system comprises: a network communication interface to receive healthcare studies; a memory coupled to the network communication interface to store received healthcare studies; a display screen coupled to the memory to display the received healthcare studies; and one or more processors coupled to the network connection interface, the memory and the display screen and configured to open a first healthcare study that contains at least one image and a current value of a parameter; automatically obtain, in response to opening the first healthcare study, at least one prior value of the parameter from one or more other healthcare studies that pre-date the first healthcare study; and control display on the display screen an image associated the first healthcare study with the current value and the at least one prior value.

### FIELD OF THE INVENTION

Embodiments of the present invention relate to the field of medical imaging; more particularly, embodiments of the present invention relate to displaying current and prior values from multiple healthcare studies simultaneously.

### BACKGROUND

Physicians and other medical personnel often review all of a patient's relevant clinical information when making healthcare decisions. The clinical information is typically included in healthcare studies and structured reports. These often include information about a patient's history, diagnostic reports from different domains, images, and other clinical data in electronic format.

The healthcare studies of a patient include a diagnostic imaging report that contains parameter values (e.g., measurements, readings, etc.) and images from examinations or procedures that are usually shared among physicians and clinicians to help in diagnosis and treatment.

With studies and structured reporting, comprehensive review of all related studies of a patient may be essential to assist with the patient's diagnosis. These studies may include a current study and one or more previous studies for a particular type of examination or procedure undertaken by a patient. Currently, users are required to open prior studies individually, which can be a labor intensive and time-consuming process that takes away from their patient and personal time. If a user is viewing a current study and has to refer to prior studies to determine changes that may have occurred in the time between studies, this is very cumbersome because reviewing the prior studies individually makes it difficult for the physician to see the patient's study measurements over time, which can help tell the story for diagnosis.

### SUMMARY OF THE INVENTION

A method and a system for displaying values of current and previous studies simultaneously are disclosed. In one embodiment, the system comprises: a network communication interface to receive healthcare studies; a memory coupled to the network communication interface to store received healthcare studies; a display screen coupled to the memory to display the received healthcare studies; and one or more processors coupled to the network connection interface, the memory and the display screen and configured to open a first healthcare study that contains at least one image and a current value of a parameter; automatically obtain, in response to opening the first healthcare study, at least one prior value of the parameter from one or more other healthcare studies that pre-date the first healthcare study; and control display on the display screen an image associated the first healthcare study with the current value and the at least one prior value.

### BRIEF DESCRIPTION OF THE DRAWINGS

The present invention will be understood more fully from the detailed description given below and from the accompanying drawings of various embodiments of the invention, which, however, should not be taken to limit the invention to the specific embodiments, but are for explanation and understanding only.

FIG. 1 illustrates an exemplary a medical information computing system environment, with which embodiments of the present invention may be implemented.

FIG. 2 is a block diagram showing one embodiment of a computing system architecture for displaying study healthcare study information (e.g., images, graphs, parameter values, etc.) in a graphical user interface (GUI).

FIG. 3 is a data flow diagram of one embodiment of a layout generation process to display current parameter (e.g., measurement) values from a current study with one or more prior values from previously generated studies with a medical image management system.

FIG. 4A illustrates an example table that is generated by a layout generator and displayed in a graphical user interface (GUI).

FIG. 4B illustrates an example of a displayed layout having the slider bar.

FIGS. 5A-5D illustrate user interfaces having example graphs generated using current and prior values of parameter data obtained from studies.

FIG. 6A is a flow diagram of one embodiment of a process for obtaining and displaying current and previous values of parameters on a display screen simultaneously.

FIG. 6B illustrates one embodiment of a process to identify previously created studies containing prior values of the parameter.

FIG. 7 illustrates an exemplary embodiment of a logical representation of a medical imaging and information management system that generates and renders images from healthcare studies.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

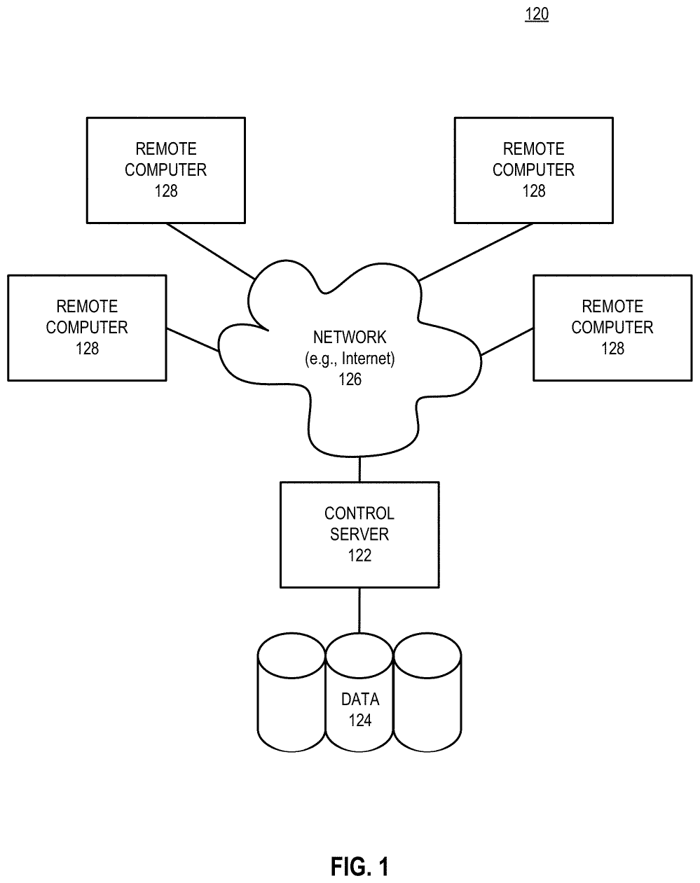

Embodiments of the present invention are directed to systems, methods, and GUIs for rendering and displaying current and prior parameter (e.g., measurement) values of current and prior healthcare studies on a display device. This helps physicians and other medical personal by providing historical data of a patient on a display device with the current data simultaneously to facilitate and potentially improve the diagnosis process. Having briefly described an overview of the present invention, embodiments of the invention will be discussed with reference to FIGS. 1-7.

The subject matter of embodiments of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies.

Having briefly described embodiments of the present invention, an exemplary operating environment suitable for use in implementing embodiments of the present invention is described below.

Referring to the drawings in general, and initially to FIG. 1 in particular, a medical information computing system environment, with which embodiments of the present invention may be implemented is illustrated and designated generally as reference numeral 120. It will be understood and appreciated by those of ordinary skill in the art that the illustrated medical information computing system environment 120 is merely an example of one suitable computing environment and is not intended to suggest any limitation as to the scope of use or functionality of the invention. Neither should the medical information computing system environment 120 be interpreted as having any dependency or requirement relating to any single component or combination of components illustrated therein.

Embodiments of the present invention may be operational with numerous general-purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, programmable consumer electronics, network PCs, minicomputers, mainframe computers, distributed computing environments that include any of the above-mentioned systems or devices, and the like.

Embodiments of the present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in association with local and/or remote computer storage media including, by way of example only, memory storage devices.

With continued reference to FIG. 1, the exemplary medical information computing system environment 120 includes a general-purpose computing device in the form of a control server 122. Components of the control server 122 may include, without limitation, a processing unit, internal system memory, and a suitable system bus for coupling various system components, including database cluster 124, with the control server 122. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 122 typically includes therein, or has access to, a variety of computer-readable media, for instance, database cluster 124. Computer-readable media can be any available media that may be accessed by server 122, and includes volatile and nonvolatile media, as well as removable and non-removable media. By way of example, and not limitation, computer-readable media may include computer storage media. Computer storage media may include, without limitation, volatile and nonvolatile media, as well as removable and non-removable media implemented in any method or technology for storage of information, such as computer-readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 122. By way of example, and not limitation, communication media includes wired media such as a wired network or direct-wired connection, and wireless media such as acoustic, RF, infrared, and other wireless media. Combinations of any of the above also may be included within the scope of computer-readable media.

The computer storage media discussed above and illustrated in FIG. 1, including database cluster 124, provide storage of computer-readable instructions, data structures, program modules, and other data for the control server 122. The control server 122 may operate in a computer network 126 using logical connections to one or more remote computers 128. Remote computers 128 may be located at a variety of locations in a medical or research environment, for example, but not limited to, clinical laboratories (e.g., molecular diagnostic laboratories), hospitals and other inpatient settings, veterinary environments, ambulatory settings, medical billing and financial offices, hospital administration settings, home health care environments, and clinicians' offices. Clinicians may include, but are not limited to, a treating physician or physicians, specialists such as intensivists, surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, veterinarians, students, and the like. The remote computers 128 may also be physically located in non-traditional medical care environments so that the entire health care community may be capable of integration on the network. The remote computers 128 may be personal computers, servers, routers, network PCs, peer devices, other common network nodes, or the like, and may include some or all of the elements described above in relation to the control server 122. The devices can be personal digital assistants or other like devices.

Exemplary computer networks 126 may include, without limitation, local area networks (LANs) and/or wide area networks (WANs). Such networking environments are commonplace in offices, enterprise-wide computer networks, intranets, and the Internet. When utilized in a WAN networking environment, the control server 122 may include a modem or other means for establishing communications over the WAN, such as the Internet. In a networked environment, program modules or portions thereof may be stored in association with the control server 122, the database cluster 124, or any of the remote computers 128. For example, and not by way of limitation, various application programs may reside on the memory associated with any one or more of the remote computers 128. It will be appreciated by those of ordinary skill in the art that the network connections shown are exemplary and other means of establishing a communications link between the computers (e.g., control server 122 and remote computers 128) may be utilized.

In operation, a clinician may enter commands and information into the control server 122 or convey the commands and information to the control server 122 via one or more of the remote computers 128 through input devices, such as a keyboard, a pointing device (commonly referred to as a mouse), a trackball, or a touch pad. Other input devices may include, without limitation, microphones, scanners, or the like. Commands and information may also be sent directly from a remote healthcare device to the control server 122. In addition to a monitor, the control server 122 and/or remote computers 128 may include other peripheral output devices, such as speakers and a printer.

Although many other internal components of the control server 122 and the remote computers 128 are not shown, those of ordinary skill in the art will appreciate that such components and their interconnection are well known. Accordingly, additional details concerning the internal construction of the control server 122 and the remote computers 128 are not further disclosed herein.

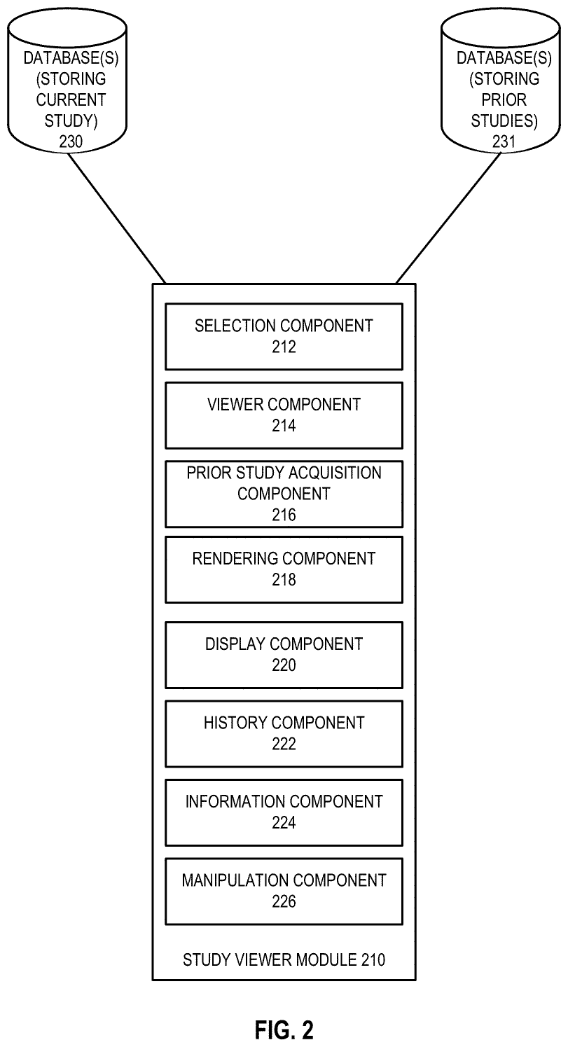

With reference to FIG. 2, a block diagram is illustrated that shows an exemplary computing system architecture for simultaneous viewing of current and prior values of parameters from healthcare studies on a display screen. It will be appreciated that the computing system architecture shown in FIG. 2 is merely an example of one suitable computing system and is not intended as having any dependency or requirement related to any single module/component or combination of modules/components.

The computing system includes a study viewer 210 and one or more databases 230 and 231 storing and maintaining current and prior healthcare studies. These studies include some current and prior studies related to the same medical procedure for the same modality. In one embodiment, the healthcare studies include images and values of one or more medical parameters (e.g., measurements, etc.) related to the healthcare study. Exemplary medical images include radiology images, laboratory images, pictures, cardiology images, such as ECHO images, and other healthcare images. One of skill in the art will appreciate that the databases may be maintained separately or may be integrated. Databases 230 may contain images or other study data (e.g., parameter values (e.g., measurements) that are linked to a patient's electronic medical record (EMR), such that images and/or parameter values may be selected from within the EMR and displayed within a viewer via the viewer 210. As utilized herein, the acronym “EMR” is not meant to be limiting, and may broadly refer to any or all aspects of the patient's medical record rendered in a digital format. Generally, the EMR is supported by systems configured to co-ordinate the storage and retrieval of individual records with the aid of computing devices. As such, a variety of types of healthcare-related information may be stored and accessed in this way. By way of example, the EMR may store one or more of the following types of information: patient demographic; medical history (e.g., examination and progress reports of health and illnesses); medicine and allergy lists/immunization status; laboratory test results, radiology images (e.g., X-rays, CTs, MRIs, etc.); other images; evidence-based recommendations for specific medical conditions; a record of appointments and physician's notes; billing records; and data received from an associated medical device. Accordingly, systems that employ EMRs reduce medical errors, increase physician efficiency, and reduce costs, as well as promote standardization of healthcare.

Display component 220 includes a graphical display device that may be a monitor, computer screen, project device or other hardware device for displaying output capable of displaying graphical user interfaces.

Study viewer 210 receives and displays images that are sourced from more than one source, or database and parameter values from the studies. Thus, a single storage repository or a single PACS system is not required. Study viewer 210 may reside on one or more computing devices, such as, for example, the control server 122 described above with reference to FIG. 1. By way of example, the control server 122 includes a computer processor and may be a server, personal computer, desktop computer, laptop computer, handheld device, mobile device, consumer electronic device, or the like.

Study viewer 210 comprises selection component 212, viewer component 214, image processing and rendering component 216, subscription component 218, and display component 220. In various embodiments, study viewer 210 includes a history component 222, an information component 224, and a manipulation component 226. It will be appreciated that while study viewer 210 is depicted as receiving healthcare studies (e.g., healthcare images and parameter values from a current study) stored in databases 230 and 231, study viewer 210 may receive healthcare studies from multiple sources including databases spread across multiple facilities and/or multiple locations. It will also be appreciated that study viewer 210 may receive healthcare studies with their images and/or parameter values from the sources described above via links within a patient's EMR.

The selection component 212 receives a selection of a healthcare study. In one embodiment, the healthcare study is the most recent study for the patient for a particular medical procedure and comprises one or more series of images and one or more parameter values (e.g., measurements, findings, impressions, patient demographics and history/risk factors, etc.). In one embodiment, each series comprises one or more images depicting the subject of the image from various angles. A list perspective within a multimedia manager provides a list of available studies, images, and other media. A clinician can select the desired items to launch in the viewer. In one embodiment, the selection of desired items may be made within the EMR.

Once the selection component 212 receives the clinician's selection, the viewer component 214 launches the viewer for the selected study. In one embodiment, when selection component 212 launches the view selected study, prior study acquisition component 216 obtains previously created studies for the patient for the same procedure in order to obtain prior parameter values that may be contained in such studies for display with the corresponding parameter values in the currently selected study.

The rendering component 218 access parameter values from the current and prior studies and generates a graphical user interface (GUI) that depicts both the current parameter value and at least one prior parameter value. This allows a physician to compare the current and prior values of a parameter for performing a diagnosis or to see a trend with respect to a medical condition associated with the patient.

The display component 220 displays the GUI generated by the rendering component 218 with the current and prior parameter values. In one embodiment, these are displayed with one or more images of the current study in the viewer along with parameter values of the current study and one or more prior studies. In one embodiment, one or more graphs illustrating the trend of current and prior values for one or more parameters are also displayed on the display screen.

In one embodiment, a history component 222 displays a history of different studies and clinical images associated with the more than one healthcare image. The history component 222 further allows a selection of one or more images from the history to be displayed in the viewer by the display component 220. For example, the selection component 212 may have received a selection from the clinician of a particular study. However, once the display component 220 has displayed the images that comprise that selected study, the history component 222 may display other studies and clinical images that are of particular interest to the clinician. The clinician may then select additional items from the history to launch within the viewer.

In one embodiment, information component 224 displays additional information associated with the more than one healthcare image, the history, or a combination thereof. The additional information comprises patient identifying information, image related information, study related information, or a combination thereof. Such additional information may also include time related information.

In one embodiment, a manipulation component 226 allows a clinician to manipulate a display of a healthcare image. For example, a clinician may determine that the image as it is rendered within the viewer is not large enough to see a desired level of detail. The clinician may zoom in or out and the manipulation component 226 manipulates the display of the image accordingly. Similarly, the clinician may desire to pan an image and the manipulation component 226 manipulates the image display accordingly.

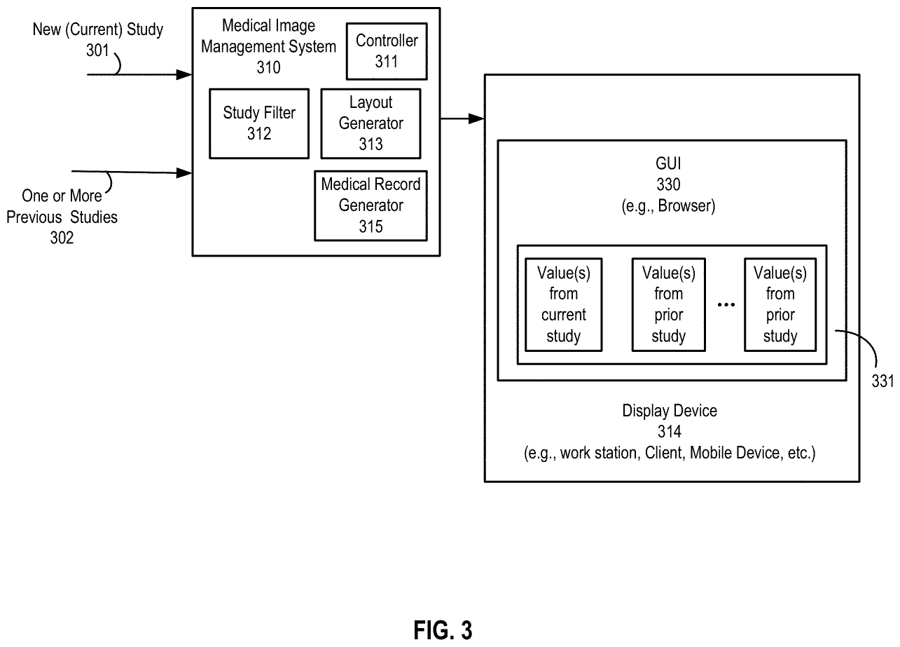

FIG. 3 is a data flow diagram of one embodiment of a layout generation process to display current parameter (e.g., measurement) values from a current study with one or more prior values from previously generated studies with a medical image management system. Medical image management system 310 allows current and prior parameter values to be accessed, graphed and/or displayed in a graphical user interface 330.

Controller 311 controls operations of medical image management system 310. In one embodiment, controller 311 comprises one or more processors, microcontrollers, and/or a combination of hardware, software and/or firmware. In one embodiment, controller 311 responds to user inputs to cause medical image management system 310 to open a current study that contains at least one current value of a parameter, automatically obtain at least one prior value of the parameter from one or more other prior healthcare studies and control display on the display screen of the system to display the current value of the parameter and the at least one prior value on the display screen at the same time. In one embodiment, controller 311 is able to cause medical image management system 310 to generate graphs (e.g., trend line graphs) and/or create medical records containing current and prior values of parameters and/or graphs containing the same.

Referring to FIG. 3, a new, or current, study 301 of a patient is received by a medical image management system 310 along with one or more previously-created patient studies 302. In one embodiment, current study 301 is sent from a medical imaging modality that performs medical imaging (e.g., cardiovascular (CV), X-ray radiography, magnetic resonance imaging, ultrasound, endoscopy, tactile imaging, thermography, nuclear medicine functional imaging techniques such as positron emission tomography (PET) and single-photon emission computed tomography (SPECT), etc.). In another embodiment, current study 301 is received from a remotely located image repository (e.g., a picture archiving and communication system (PACS)).

Previously-created studies 302 are obtained from the local storage of medical image management system 310 or one or more remotely located image repositories (e.g., a PACS). Previously-created studies 302 may be obtained in response to a user of medical image management system 310 signaling the medical image management system 310 to obtain such studies for the layout generation process.

In one embodiment, study filter 312 filters one or more previously generated studies to determine which studies are for the same patient as current study 301 and for the same procedure in order to obtain prior values of parameters that are relevant to the parameters that are part of current study 301. In one embodiment, study filter 312 identifies the prior study that is closest in time to current study 301.

After obtaining the appropriate previously generated studies, layout generator 313 obtains the current values of parameters from current study 301 and prior values from one or more prior studies and generates a layout 331 in a GUI (or viewer) 330 on display device 314. In one embodiment, layout 331 shows current parameter values next to prior parameter values. This allows a physician or other medical profession to easily compare prior study values with current study values. In one embodiment, layout generator 331 generates a side-by-side layout with the parameter values of current study 301 next to parameter values from the one or more previous-created studies 302, such as shown in window 331.

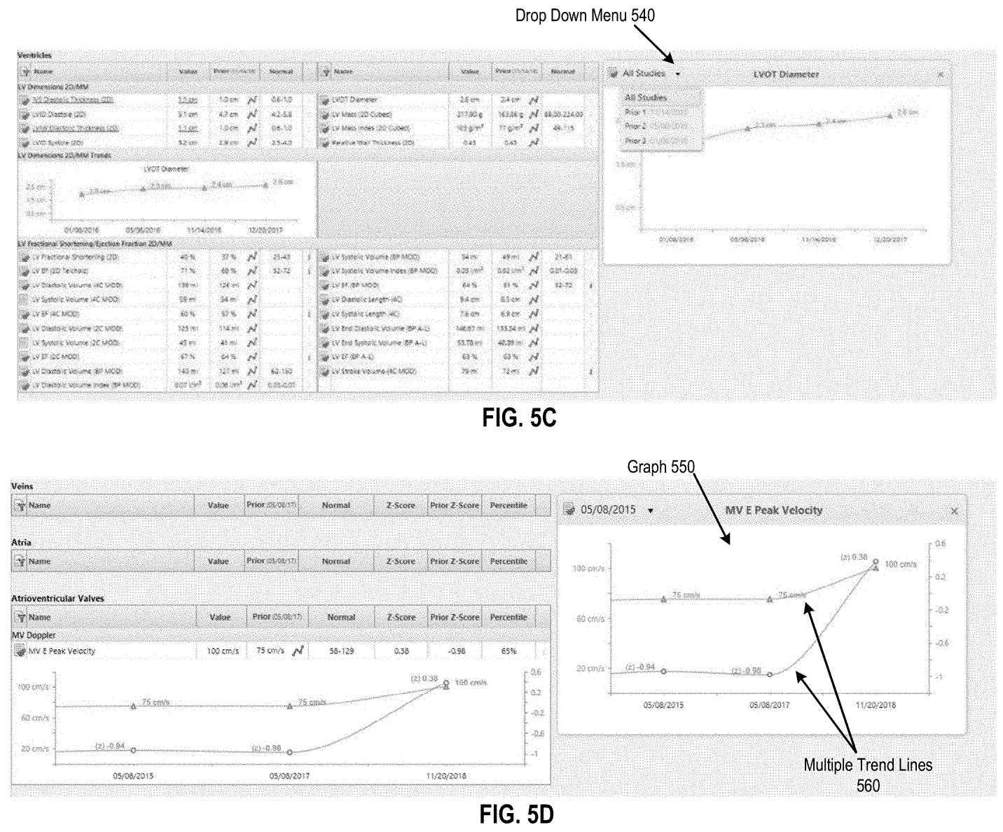

In one embodiment, layout generator 313 also generates graphs that chart the prior and current values of a parameter. In one embodiment, the graphs are trend line graphs. In one embodiment, the graphs are displayed in GUI 330. In one embodiment, the graphs are displayed in response to a user input made with respect to the display screen. In one embodiment, the user input comprises a selection of a trend icon on the display screen. In one embodiment, a chart or table of the current and prior values of each parameter are displayed in GUI 330 along with the graphs. Example graphs are discussed below in conjunction with FIGS. 5A-5D.

In one embodiment, layout generator 313 displays an amount of change (e.g., a percentage change) indication indicative of an amount of change that has occurred between the current value and the at least one prior value in response to receiving a user input with respect to the display screen. In one embodiment, the user input comprises movement of a cursor on the display screen to hover over a display location displaying the current value.

In one embodiment, layout generator 313 generates a layout (e.g., a graph) showing where the current parameter value and/or prior parameter values with respect to a range of the parameter value considered normal. Thus, upon review of the layout, an individual can determine whether the current or prior parameter values are in the normal range. In one embodiment, the layout comprises a graph with an area (e.g., color band) indicating the normal parameter value range.

In one embodiment, medical record generator 315 embeds one or more graphs generated by layout generator 313 into a medical record.

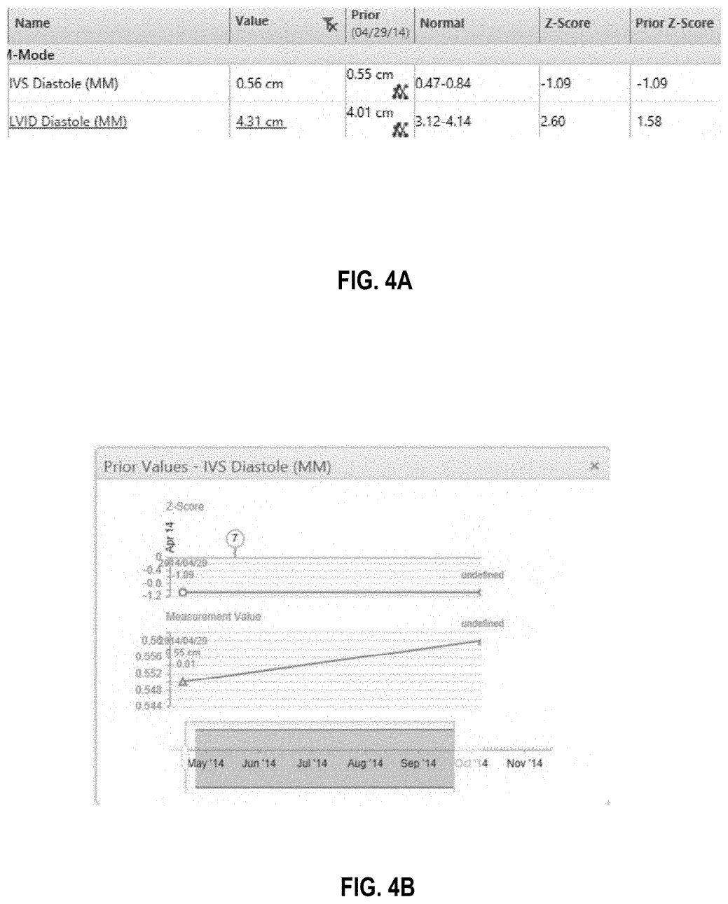

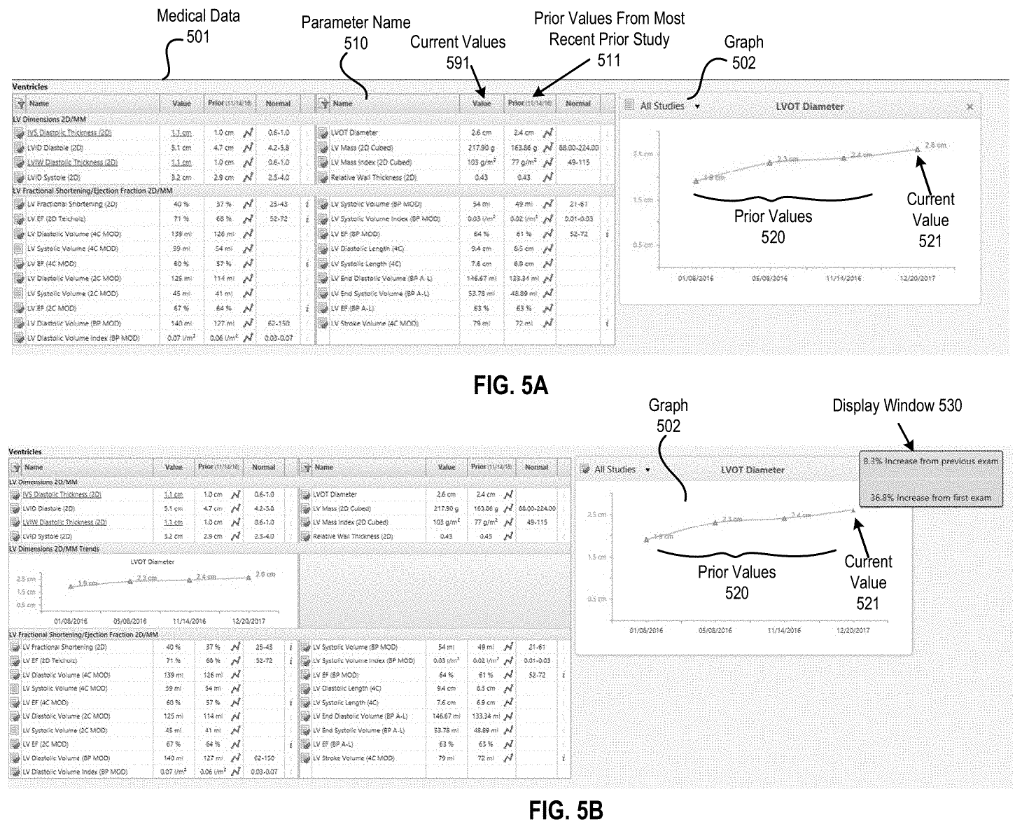

In one embodiment, medical image management system 310 displays the prior value and Z-score next to the current values for the study. FIG. 4A illustrates an example table that is generated by layout generator 313 and displayed in GUI 330.

Referring to FIG. 4A, in one embodiment, the prior columns are titled “Prior” and “Prior Z-Score” and only appear when a pertinent prior study is available. If multiple prior studies are available, the first prior study finalized with similar procedure type will be displayed. A “similar” study is defined by procedure performed, and the current report pulls from the same “pertinent study”.

In one embodiment, for parameters (e.g., measurements) having a prior value available, layout generator 313 generates a trending icon that appears in GUI 330. Once selected, a trending graph appears displaying the measurement values and Z-scores over time. In one embodiment, the trending graph has the first studies value displayed on the y-axis and each new value being displayed to the right from every previous study. In one embodiment, a slider bar is displayed along the bottom, allowing the user to shorten the timeline, but the current study's value always appears. In one embodiment, the trending control does require at least two data points before the icon will display and will be in a separate dialog window. FIG. 4B illustrates an example of a displayed layout having the slider bar.

FIGS. 5A-5D illustrate user interfaces having example graphs generated using current and prior values of parameters obtained from current and prior studies, respectively. In one embodiment, these graphs are from cardiovascular (CV) studies. However, the techniques described herein are not limited to use with CV studies. The user interfaces are generated by a medical image management system and displayed on the display screen of the system.

Referring to FIG. 5A, the user interface includes a section displaying medical data 501 that contains a number of parameters with both current and prior values. These parameters include measurements and/or calculated values. In one embodiment, there are three columns, one for the parameter name or identifier, one for the current values and one for prior values for each parameter from the most recent prior study. For example, column 510 shows the names of a number of CV parameters, column 591 illustrates the current value from the current study for each of the parameters in column 510, and column 511 illustrates the prior value from most recent prior study for each of the parameters in column 510.

FIG. 5A also includes a graph area 502 that is able to display a graph associated with each selected parameter in one of the parameter columns such as, for example, column 510. In the example, a graph of current and prior LVOT diameter measurement values are shown. Specifically, prior values 520 include three LVOT diameter measurement values and a current LVOT diameter measurement value 521. The points in the graph representing the current and prior values are connected to show a multi-point trend line for the parameter.

FIG. 5B illustrates one embodiment of a user interface in which the percentage change and/or percentage increase (or decrease) is depicted for the graphed values. Referring to FIG. 5B, graph area 502 depicted a graph of a LVOT diameter measurement values from three prior studies and the one value, current value 521, from the current study. In one embodiment, when the user hovers the cursor over current value 521, the system generates and displays display window 530 on the display screen which displays a percentage change (e.g., increase, decrease) between current value 521 and the prior value from the most recent prior study. A percentage change (e.g., increase, decrease) between current value 521 and the prior value from first prior study containing that parameter value is also displayed in display window 530. Note that in other embodiments only one percentage change is shown. In one embodiment, the hover capability only occurs in the graph area 502 when there are at least 2 studies (the current study and at least 1 prior study).

Alternatively, display window 530 may display percentage changes between all values. Also, in an alternative embodiment, display window 530 provides an alert or other notification that is based on whether current value 521 or the percentage change being displayed warrants action or attention, is normal or not normal. In yet another alternative embodiment, a user can select a prior value (e.g., clicking on the prior value with a cursor), which causes the loading of the prior study and image on which the measurement was performed. The prior value that is selected may be in a graph shown in a graph area (e.g., graph area 502) or in a column of prior values (e.g., column 511). FIG. 5C illustrates a user interface with a multiple point trend and an embedded graph. Referring to FIG. 5C, the user interface includes drop down menu 540 that allows a user to select whether the embedded graph includes prior values of a parameter from all prior studies or a subset of prior studies along with the current value of a parameter.

FIG. 5D illustrates a user interface in which the graph area shows multiple trend lines of prior and current parameter values. Referring to FIG. 5D, graph area 550 shows two trend lines 560 with current and prior values of two different parameters.

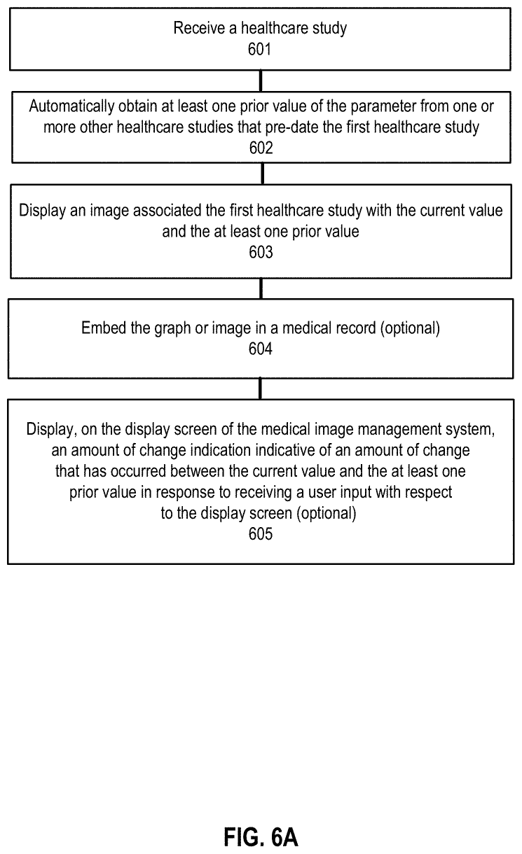

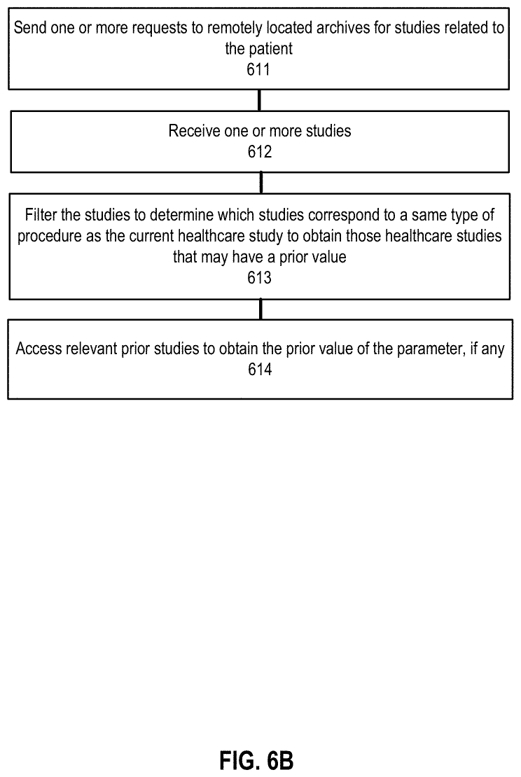

FIG. 6A is a flow diagram of one embodiment of a process for obtaining and displaying current and previous values of parameters on a display screen simultaneously. In one embodiment, the processes are performed by processing logic that may comprise hardware (circuitry, dedicated logic, etc.), software (e.g., software running on a chip), firmware, or a combination of the three. In one embodiment, the process is performed by a medical image management system.

Referring to FIG. 6A, the process beings by receiving a healthcare study (processing block 601). In one embodiment, the healthcare study is sent to the medical image management system by a modality that creates the study. In an alternative embodiment, the healthcare study is sent from a medical image archive, such as, for example, a picture archiving and communication system (PACS) or other remotely-located storage facility. In one embodiment, the healthcare study is sent in response to a request. In an alternative embodiment, the healthcare study is sent automatically. In one embodiment, the healthcare study is received via a network interface and stored in a memory of the medical image management system.

In one embodiment, a first healthcare study that contains at least one image and a current value of one or more parameters. The parameters may comprise a measurement made by an operator, a physician, and/or other medical personnel when creating the study (e.g., operating a modality) or by a physician, and/or other medical personnel made in response to reviewing images or other output data from the modality.

After the healthcare study has been obtained, processing logic automatically obtains at least one prior value of the parameter from one or more other healthcare studies that pre-date the first healthcare study (processing block 602).

In one embodiment, only a single prior value of the parameter is obtained. In one embodiment, this single prior value is more recent value of the parameter from the healthcare study for the same medical procedure (e.g., is a cardiovascular (CV) study, etc.) that is closest in time to the healthcare study currently being viewed. In another embodiment, multiple or all prior values for the same parameter from previous healthcare studies for the same procedure are obtained. FIG. 6B illustrates one embodiment of a process to identify previously created studies containing prior values of the parameter. In one embodiment, the processes are performed by processing logic that may comprise hardware (circuitry, dedicated logic, etc.), software (e.g., software running on a chip), firmware, or a combination of the three. In one embodiment, the process is performed by a medical image management system. Referring to FIG. 6B, the process to identify prior values begins by sending one or more requests to remotely located archives for studies related to the patient (processing block 611). In one embodiment, the medical image management system sends requests its network communication interface to one or more remotely-located storage facilities (e.g., PACS). In one embodiment, the requests include patient identification information (e.g., patient identifier (ID), patient name, etc.).

In response to the requests, processing logic receives one or more studies (processing block 612) and filters the studies to determine which studies correspond to a same type of procedure as the current healthcare study to obtain those healthcare studies that may have a prior value (processing block 613). In one embodiment, the filtering includes determining whether each receives study is for the patient associated with the current healthcare study. This is necessary in cases where the patient identification information may cause studies from multiple patients to be retrieved. For example, if the patient has a common last name and only an initial of their first name is available, all studies for patient's last name and a first name starting with the same initial may be retrieved. These studies would have to be filtered to obtain the study for the same procedure and the same patient.

Once the healthcare studies that may contain a prior value of the parameter have been identified, processing logic access those studies to obtain the prior value of the parameter, if any (processing block 614).

Referring back to FIG. 6A, in one embodiment, the prior values are obtained in response to opening the healthcare study. In another embodiment, the prior values are obtained as soon as the study is obtained and opened and one or more images series in the study are accessed and/or displayed. In yet another embodiment, the prior values are obtained in response to a message displayed on the display screen of the medical image management system asking the user whether they wish to obtain such values from earlier studies of the patient. In still yet another embodiment, the prior values are obtained in response to a user selecting a graphical user interface (GUI) element on the display screen (e.g., prior values icon or button, drop-down menu option, etc.).

After obtaining the prior value(s) for the parameter, processing logic displays an image associated the first healthcare study with the current value and the at least one prior value (processing block 603). In one embodiment, the current value of the parameter and at least one prior value of the parameter are displayed in proximity to each other on the display screen. In one embodiment, the current value and all prior values are displayed in proximity to each other on the display screen. In one embodiment, the display screen displays a table or chart containing the current values and one or more prior values. In one embodiment, the display screen displays the current value of the parameter and at least one prior value of the parameter adjacent to each other.

In one embodiment, the image is a graph on the display screen depicting the current value and one or more prior values. In one embodiment, the graph illustrates a trend line associated with the current value and the at least one prior value. In one embodiment, the graph is displayed in response to a user input made with respect to the display screen. In one embodiment, the user input comprises a selection of a trend icon on the display screen. In another embodiment, the user input comprises a selection of a drop-down menu option on the display screen.

Note that other selection mechanisms and GUI elements may be used to cause the system to display the graph or other image associated with the current healthcare study.

In one embodiment, processing logic embeds the graph or image in a medical record (processing block 604). For example, in one embodiment, selection of the graph depicting both current and one or more prior values is selected and embedded in a patient EMR record. The graph may depict a trend line.

In one embodiment, the current value is displayed in a manner that indicates its relationship with respect to whether the current value is inside or outside a range considered normal. For example, the current value may be displayed on the display screen along with a typical range of values for the parameter (e.g., a band of values of the parameter that are typical for individuals, etc.).

In one embodiment, processing logic displays, on the display screen of the medical image management system, an amount of change indication indicative of an amount of change that has occurred between the current value and the at least one prior value in response to receiving a user input with respect to the display screen (processing block 605). In one embodiment, this is optional. In one embodiment, the user input comprises movement of a cursor on the display screen to hover over a display location displaying the current value.

### An Exemplary Medical Imaging Management System

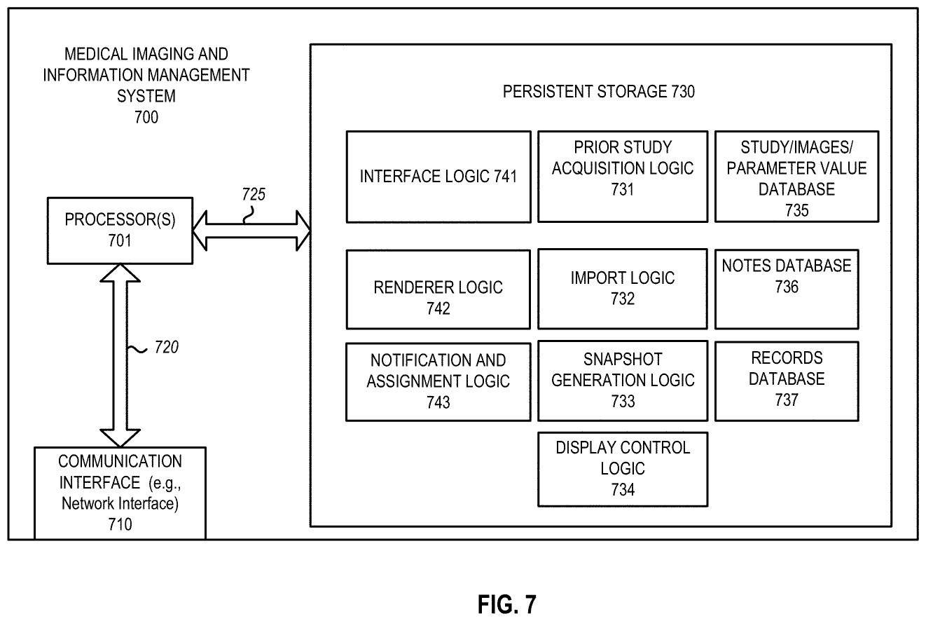

FIG. 7 illustrates an exemplary embodiment of a logical representation of a medical imaging and information management system 700 that generates and renders layouts with current and prior values of parameters discussed above. In one embodiment, system 700 is part of a medical image system such as detailed above.

The medical imaging and information management system 700 includes one or more processors 701 that are coupled to communication interface logic 710 via a first transmission medium 720. The communication interface logic 710 enables communications with other electronic devices, specifically enabling communication with remote users such as doctors, nurses and/or medical technicians, remote databases (e.g., PACS) that store healthcare studies, and healthcare modalities that generate and send studies. According to one embodiment of the disclosure, communication interface logic 710 may be implemented as a physical interface including one or more ports for wired connectors. Additionally, or in the alternative, communication interface logic 710 may be implemented with one or more radio units for supporting wireless communications with other electronic devices.

The processor(s) 701 is further coupled to persistent storage 730 via transmission medium 725. According to one embodiment of the disclosure, persistent storage 730 may include (a) user interface logic 741, (b) rendering logic 742, (c) notification and assignment logic 743, (d) prior study acquisition logic 731, (e) an import logic 732, (f) a snapshot generation logic 733, (g) a display control logic 734, (h) an image and parameter value database 735, (i) a notes database 736 and (j) a records database 737.

The user interface logic 741 may include logic for enabling interaction between a user and the display areas being displayed on the display screen.

The rendering logic 742 includes logic for requesting parameter values and images from current and prior studies to be rendered as described above. In one embodiment, the rendering logic 742 performs one or more processing operations on the current and prior parameter values, such as, for example, those described above including calculating percentage increase and decrease changes of parameter values between a current healthcare study and one or more prior healthcare studies of the patient, as well as displaying the percentage difference between the immediately prior examination and the first examination.

The prior study acquisition logic 731 includes logic for requesting and receiving prior studies of a patient for the same medical procedure associated with the current study being selected and/or displayed by a user with the medical imaging and information management system 700. In one embodiment, prior study acquisition logic 731 performs the operations associated and described in conjunction with FIG. 6B.

The notification and assignment logic 743 includes logic to issue and send notifications and/or assignments for study reviews to one or more of physicians and medical personnel.

The import logic 732 may include logic for retrieving one or more pieces of information from a storage device and importing each of the one or more pieces of information into a separate display area of a viewer or viewer template. For example, the pieces of information may include, but are not limited or restricted to, (i) parameter values from current and prior studies; (ii) medical images, including x-rays, mammograms, computerized tomography (CT) scans, magnetic resonance imaging (MRI), positron emission tomography (PET) scan and/or ultrasound imaging, (iii) physician's notes regarding one or more of the medical images and/or (iv) medical records corresponding to one or more of the subjects of the one or more medical images.

The snapshot generation logic 733 includes logic for saving at least a first state of the layout template. Saving the first state may include storing, at least, (i) the one or more pieces of information, and (ii) viewing properties of each of the one or more pieces of information in a non-transitory computer-readable medium. The layout template may depict a layout depicting one or more parameter values from a current study and one or more parameter values from one or more previously-created studies.

The display control logic 734 includes logic for displaying graphs and images that have been rendered locally as well as parameter values as discussed above. In one embodiment, the display control logic 734 includes logic to display a browser into which the images, graphs, and parameter values are displayed.

The images and parameter values database 735, the notes database 736 and the records database 737 may comprise a single non-transitory computer-readable medium storage device or may each be a separate non-transitory computer-readable medium storage device. The images database 735 stores parameter values and medical images that a user may import into a display area of a viewer or other GUI. The notes database 736 stores notes recorded by a doctor, nurse, medical technician, etc., that a user may import into a display area of a layout template. Finally, the records database 737 stores medical records that a user may import into a display area of a layout template.

There is a number of example embodiments described herein.

Example 1 is a system comprising: a network communication interface to receive healthcare studies; a memory coupled to the network communication interface to store received healthcare studies; a display screen coupled to the memory to display the received healthcare studies; and one or more processors coupled to the network connection interface, the memory and the display screen and configured to open a first healthcare study that contains at least one image and a current value of a parameter; automatically obtain, in response to opening the first healthcare study, at least one prior value of the parameter from one or more other healthcare studies that pre-date the first healthcare study; and control display on the display screen an image associated the first healthcare study with the current value and the at least one prior value.

Example 2 is the system of example 1 that may optionally include that at least one prior value is one prior value from one healthcare study of the one or more healthcare studies that is closest in time to the first healthcare study.

Example 3 is the system of example 2 that may optionally include that the current value and one prior value are displayed in proximity to each other on the display screen.

Example 4 is the system of example 1 that may optionally include that the processor is further operable to identify the one or more studies by sending one or more requests to remotely located archives for studies related to the patient; receiving at least one study in response to the one or more requests; filtering the at least one study to determine which studies correspond to a same type of procedure as the first healthcare study to obtain the one or more other healthcare studies.

Example 5 is the system of example 4 that may optionally include that the same type of study is a cardiovascular (CV) study.

Example 6 is the system of example 1 that may optionally include that the processor is further operable to display on the display screen an amount of change indication indicative of an amount of change that has occurred between the current value and the at least one prior value in response to receiving a user input with respect to the display screen.

Example 7 is the system of example 6 that may optionally include that the user input comprises movement of a cursor on the display screen to hover over a display location displaying the current value.

Example 8 is the system of example 1 that may optionally include that the current value is displayed in a manner that indicates its relationship with respect to whether the current value is inside or outside a range considered normal.

Example 9 is the system of example 8 that may optionally include that the processor is further operable to display a graph on the display screen depicting the current value and the at least one prior value.

Example 10 is the system of example 8 that may optionally include that the graph illustrates a trend line associated with the current value and the at least one prior value.

Example 11 is the system of example 10 that may optionally include that the processor is operable to display the graph in response to a user input made with respect to the display screen.

Example 12 is the system of example 11 that may optionally include that the user input comprises a selection of a trend icon on the display screen.

Example 13 is the system of example 10 that may optionally include that the processor is further operable to embed the graph in a medical record.

Example 14 is the system of example 1 that may optionally include that the current value and at least one prior value comprise measurements.

Example 15 is a method comprising: opening a first healthcare study that contains at least one image and a current value of a parameter; automatically obtaining, in response to opening the first healthcare study, at least one prior value of the parameter from one or more other healthcare studies that pre-date the first healthcare study; and displaying on a display screen an image associated the first healthcare study with the current value and the at least one prior value.

Example 16 is the method of example 15 that may optionally include that the at least one prior value is one prior value from one healthcare study of the one or more healthcare studies that is closest in time to the first healthcare study.

Example 17 is the method of example 15 that may optionally include identifying the one or more studies by sending one or more requests to remotely located archives for studies related to the patient; receiving at least one study in response to the one or more requests; filtering the at least one study to determine which studies correspond to a same type of procedure as the first healthcare study to obtain the one or more other healthcare studies.

Example 18 is the method of example 17 that may optionally include displaying an amount of change indication indicative of an amount of change that has occurred between the current value and the at least one prior value in response to receiving a user input with respect to the display, wherein the user input comprises movement of a cursor on the display to hover over a display location displaying the current value.

Example 19 is the method of example 15 that may optionally include that the current value is displayed in a manner that indicates its relationship with respect to whether the current value is inside or outside a range considered normal.

Example 20 is the method of example 15 that may optionally include that the image comprises a graph depicting the current value and the at least one prior value.

Example 21 is the method of example 20 that may optionally include that the graph illustrates a trend line associated with the current value and the at least one prior value.

Example 22 is the method of example 21 that may optionally include embedding the graph in a medical record.

Example 23 is the method of example 15 that may optionally include opening and displaying one study of the one or more prior studies in response to selection of one prior value of the one or more prior values.

Example 24 is a non-transitory computer readable storage media having instructions stored thereupon which, when executed by a system having at least a processor, a memory and a display screen therein, cause the system to perform a method comprising: opening a first healthcare study that contains at least one image and a current value of a parameter; automatically obtaining, in response to opening the first healthcare study, at least one prior value of the parameter from one or more other healthcare studies that pre-date the first healthcare study; and displaying on a display screen an image associated the first healthcare study with the current value and the at least one prior value.

Example 25 is the non-transitory computer readable storage media of example 24 that may optionally include that the at least one prior value is one prior value from one healthcare study of the one or more healthcare studies that is closest in time to the first healthcare study.

Example 26 is the non-transitory computer readable storage media of example 24 in which the method may optionally include identifying the one or more studies by sending one or more requests to remotely located archives for studies related to the patient; receiving at least one study in response to the one or more requests; filtering the at least one study to determine which studies correspond to a same type of procedure as the first healthcare study to obtain the one or more other healthcare studies.

Example 27 is the non-transitory computer readable storage media of example 26 in which the method may optionally include displaying an amount of change indication indicative of an amount of change that has occurred between the current value and the at least one prior value in response to receiving a user input with respect to the display, wherein the user input comprises movement of a cursor on the display to hover over a display location displaying the current value.

Example 28 is the non-transitory computer readable storage media of example 24 that may optionally include that the image comprises a graph depicting the current value and the at least one prior value.

Example 29 is the non-transitory computer readable storage media of example 28 that may optionally include that the graph illustrates a trend line associated with the current value and the at least one prior value.

Some portions of the detailed descriptions above are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention also relates to apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus.

The algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the invention as described herein.

A machine-readable medium includes any mechanism for storing or transmitting information in a form readable by a machine (e.g., a computer). For example, a machine-readable medium includes read only memory (“ROM”); random access memory (“RAM”); magnetic disk storage media; optical storage media; flash memory devices; electrical, optical, acoustical or other form of propagated signals (e.g., carrier waves, infrared signals, digital signals, etc.); etc.

Whereas many alterations and modifications of the present invention will no doubt become apparent to a person of ordinary skill in the art after having read the foregoing description, it is to be understood that any particular embodiment shown and described by way of illustration is in no way intended to be considered limiting. Therefore, references to details of various embodiments are not intended to limit the scope of the claims which in themselves recite only those features regarded as essential to the invention.

We claim:

1. A system comprising:
a network communication interface to receive healthcare studies;
a memory coupled to the network communication interface to store received healthcare studies;
a display screen coupled to the memory to display the received healthcare studies; and
one or more processors coupled to the network connection interface, the memory and the display screen and configured to
open a first healthcare study that contains at least one image and a current value of a parameter;
automatically obtain, in response to opening the first healthcare study, at least one prior value of the parameter from one or more other healthcare studies that pre-date the first healthcare study; and
control display on the display screen an image associated the first healthcare study with the current value and the at least one prior value.

2. The system defined in claim 1 wherein the at least one prior value is one prior value from one healthcare study of the one or more healthcare studies that is closest in time to the first healthcare study.

3. The system defined in claim 2 wherein the current value and one prior value are displayed in proximity to each other on the display screen.

4. The system defined in claim 1 wherein the processor is further operable to identify the one or more studies by
sending one or more requests to remotely located archives for studies related to the patient;
receiving at least one study in response to the one or more requests;
filtering the at least one study to determine which studies correspond to a same type of procedure as the first healthcare study to obtain the one or more other healthcare studies.

5. The system defined in claim 4 wherein the same type of study is a cardiovascular (CV) study.

6. The system defined in claim 1 wherein the processor is further operable to display on the display screen an amount of change indication indicative of an amount of change that has occurred between the current value and the at least one prior value in response to receiving a user input with respect to the display screen.

7. The system defined in claim 6 wherein the user input comprises movement of a cursor on the display screen to hover over a display location displaying the current value.

8. The system defined in claim 1 wherein the current value is displayed in a manner that indicates its relationship with respect to whether the current value is inside or outside a range considered normal.

9. The system defined in claim 8 wherein the processor is further operable to display a graph on the display screen depicting the current value and the at least one prior value.

10. The system defined in claim 8 wherein the graph illustrates a trend line associated with the current value and the at least one prior value.

11. The system defined in claim 10 wherein the processor is operable to display the graph in response to a user input made with respect to the display screen.

12. The system defined in claim 11 wherein the user input comprises a selection of a trend icon on the display screen.

13. The system defined in claim 10 wherein the processor is further operable to embed the graph in a medical record.

14. The system defined in claim 1 wherein the current value and at least one prior value comprise measurements.

15. A method comprising:
opening a first healthcare study that contains at least one image and a current value of a parameter;
automatically obtaining, in response to opening the first healthcare study, at least one prior value of the parameter from one or more other healthcare studies that pre-date the first healthcare study; and
displaying on a display screen an image associated the first healthcare study with the current value and the at least one prior value.

16. The method defined in claim 15 wherein the at least one prior value is one prior value from one healthcare study of the one or more healthcare studies that is closest in time to the first healthcare study.

17. The method defined in claim 15 further comprising identifying the one or more studies by
sending one or more requests to remotely located archives for studies related to the patient;
receiving at least one study in response to the one or more requests;
filtering the at least one study to determine which studies correspond to a same type of procedure as the first healthcare study to obtain the one or more other healthcare studies.

18. The method defined in claim 17 further comprising displaying an amount of change indication indicative of an amount of change that has occurred between the current value and the at least one prior value in response to receiving a user input with respect to the display, wherein the user input comprises movement of a cursor on the display to hover over a display location displaying the current value.

19. The method defined in claim 15 wherein the current value is displayed in a manner that indicates its relationship with respect to whether the current value is inside or outside a range considered normal.

20. The method defined in claim 15 wherein the image comprises a graph depicting the current value and the at least one prior value.

21. The method defined in claim 20 wherein the graph illustrates a trend line associated with the current value and the at least one prior value.

22. The method defined in claim 21 further comprising embedding the graph in a medical record.

23. The method defined in claim 15 further comprising opening and displaying one study of the one or more prior studies in response to selection of one prior value of the one or more prior values.

24. A non-transitory computer readable storage media having instructions stored thereupon which, when executed by a system having at least a processor, a memory and a display screen therein, cause the system to perform a method comprising:
opening a first healthcare study that contains at least one image and a current value of a parameter;
automatically obtaining, in response to opening the first healthcare study, at least one prior value of the parameter from one or more other healthcare studies that pre-date the first healthcare study; and
displaying on a display screen an image associated the first healthcare study with the current value and the at least one prior value.

25. The computer readable storage media defined in claim 24 wherein the at least one prior value is one prior value from one healthcare study of the one or more healthcare studies that is closest in time to the first healthcare study.

26. The computer readable storage media defined in claim 24 further comprising identifying the one or more studies by
sending one or more requests to remotely located archives for studies related to the patient;
receiving at least one study in response to the one or more requests;
filtering the at least one study to determine which studies correspond to a same type of procedure as the first healthcare study to obtain the one or more other healthcare studies.

27. The computer readable storage media defined in claim 26 further comprising displaying an amount of change indication indicative of an amount of change that has occurred between the current value and the at least one prior value in response to receiving a user input with respect to the display, wherein the user input comprises movement of a cursor on the display to hover over a display location displaying the current value.

28. The method defined in claim 24 wherein the image comprises a graph depicting the current value and the at least one prior value.

29. The method defined in claim 28 wherein the graph illustrates a trend line associated with the current value and the at least one prior value.
